(19) United States
(12) Patent Application Publication
(10) Pub. No.: US 2018/0000399 A1
(43) Pub. Date: Jan. 4, 2018

(54) DEVICES WHICH PROMPT DIVERSE BRAIN STATES TO UPGRADE BRAIN PERFORMANCE

(71) Applicants: Margaret A. MOORE, Wellesley, MA (US); Shelley CARSON, Plymouth, MA (US); Ran ZILCA, Raanana (IL); Jerilyn ASHER, Natick, MA (US)

(72) Inventors: Margaret A. MOORE, Wellesley, MA (US); Shelley CARSON, Plymouth, MA (US); Ran ZILCA, Raanana (IL); Jerilyn ASHER, Natick, MA (US)

(21) Appl. No.: 15/545,447

(22) PCT Filed: Jan. 28, 2016
(86) PCT No.: PCT/US16/15410

§ 371 Date: Jul. 21, 2017

Related U.S. Application Data
(60) Provisional application No. 62/108,716, filed on Jan. 28, 2015.

(51) Int. Cl. A61B 5/00; G06F 3/01; A61B 5/16; G06F 19/00

(52) U.S. Cl. CPC A61B 5/4064; G06F 19/3481; G06F 3/015; A61B 5/165; A61B 5/167; A61B 5/168

(57) ABSTRACT

An electronic device that includes an electronic calendar containing scheduled events, the device further including a) software that matches an event with an optimal brain state, and b) means for convening to the user of the device such optimal brain state when the calendar indicates that the event is imminent or has arrived.

### BACKGROUND

There are optimal brain states for performing certain tasks, engaging in certain activities, responding to certain events, and optimizing brain organization, performance, health, and wellbeing. The inventors have used electronic devices to match brain states and tasks, activities, or events.

### SUMMARY OF THE INVENTION

This invention builds upon principles set out in two Harvard Health books co-authored by two of the inventors, Organize Your Mind, Organize Your Life, Hammerness and Moore (Harlequin 2012), and Your Creative Brain, Carson (Jossey-Bass 2012), both of which are hereby incorporated by reference. The invention is focused on brain organization, performance, and health using one or more devices that measure parameters such as time using calendars or time-keeping and time-tracking ability, geographical location, or user physiological or psychological parameters such as EEG, heart rate, or self-report of a particular state.

For purposes of this description, the mind is considered to be the human capacity of conscious control of certain brain functions, performance, and health in daily life.

Referring to FIG. 3, we have identified a number of brain states, non-rigid depictions of brain activity, drawing in part on the scientific literature. Each brain state can be deployed in the furtherance of a particular task or activity or event, as prompted by the electronic device or user, with graphics and instructions as needed. The brain states include:
Evaluate Brain State—a high executive control state used for tasks involving evaluating, critiquing, analyzing, or decision-making.
Self-Evaluate Brain State—a high executive control state, combined with self-referencing, used for activities involving self-evaluation.
Thinking Brain State—a high executive control state used for tasks involving the exploration, thinking through, and “figuring out” of a problem to be solved, or planning a project, or processing email, or completion of a set of tasks on a checklist.
Strategic Brain State—an agile brain state where one zooms attention out from focus on nitty gritty details and considers larger questions, contexts, patterns or systems.
Collaborate Brain State—a richly activated state of two or more people collaborating on a thinking or creative activity (see FIGS. 4-5).
Rapport Brain State—a state of connection, trust, and empathy between two or more people who are sharing positive or negative experiences (see FIGS. 6-9).
Vision Brain State—envisioning or imagining a future possibility, a state of focused and creative daydreaming.
Mind Wandering Brain State—a defocused brain state where the brain is on automatic pilot and wanders freely, like daydreaming (see FIGS. 10-11).
Flow Brain State—a partially defocused brain state used for engagement in a creative, emergent project or activity, such as a design, writing, athletic or artistic endeavor (see FIGS. 19-20).
Nonlinear Brain State—a partly defocused brain state used for playful and creative divergent activities such as brainstorming or playing games, such as charades.
Embodied Brain State—a defocused brain state where one has a “mind meld” with another person in action, learning by observing, not thinking or evaluating.
Meta-Awareness Brain State—a meta-cognitive brain state where one observes oneself, thoughts, emotions, and behaviors, in a non-judgmental, non-reactive mode, as though one is watching oneself in a movie, not being the main actor in the movie (see FIGS. 15-18).
Open Awareness Brain State—a defocused brain state when one's attentional resources are spread across several brain sensory regions which may include eyesight, hearing, smell, and touch (see FIGS. 12-14).
Narrow Awareness Brain State—a somewhat focused brain state when one's attentional resources are focused narrowly on a sensory experience such as chewing and tasting when eating, or certain muscle groups when exercising, or breathing in and out in a meditation (see FIGS. 21-22).

In a first aspect, the invention features an electronic device that has an electronic calendar containing scheduled events such as meetings, exercise sessions, social events, and medical appointments. The electronic device can be a computer, tablet, phone, watch, robot, a wearable device, or an electronic game device. Further, the device has:
1. software that matches an event with an optimal brain state for such event, and
2. means for conveying to the user of the device that optimal brain state when the calendar indicates an event reminder or that the event is imminent or has arrived.

In embodiments, the device includes a screen, and the optimal brain state is displayed visually on the screen. The device can include an audible function, and the device conveys to the user an audible prompting signal when the calendar indicates that the event is imminent or has arrived. The prompting signal includes a verbal identification of the optimal brain state and additional instructions.

The device can include means, e.g., a keyboard or touchscreen, whereby the user can input one or more of the following: i) an assessment, e.g., on a one-to-five scale, of the user's real-time physical energy or health state, ii) an assessment, e.g., on a one-to-five scale, of the user's real-time emotional state, and iii) an assessment, e.g., on a one-to-five scale, of the user's real-time cognitive performance, e.g., focus, mental agility, creativity, and working memory.

In another embodiment, the device includes means for physically measuring a physiological parameter, e.g., EEG, heart rate, heart rate variability or blood pressure, that may vary with one or more physical or emotional states, or brain performance or health, of the user; the device includes means for conveying to the user recommended brain states and actions to improve said physical or emotional states, or brain performance or health. The conveying means can be audible cues, or a screen on which can be displayed brain states and recommended actions.

The device can be further programmed to aid the user in practicing a selected desirable brain state habit by i) conveying to the user prompts, at pre-determined intervals, or based on geographic or personal metric triggers, to practice the habit, ii) tracking the practicing of the selected habit, iii) tracking the rewards flowing from the practicing of the selected habit, and iv) using the tracking of step iii) to calculate the degree to which the habit becomes automatic.

The device can be programmed to receive and store a goal of the user, attainment of which is aided by practicing and keeping a brain state habit.

The device can be programmed to present to the user a series of experiments regarding one or more desirable brain state habits, and to store the results of said experiments.

In another aspect, the invention features an electronic device that is programmed to provide visual or audible prompts to the user, the prompts occurring either: a) randomly throughout the day, or b) at predetermined times throughout the day, wherein the prompts convey to the user a predetermined brain state such as meta awareness (a “pause” brain state), open awareness (a “reboot” brain state), BQ (a “body sensory awareness” brain state) or a “mind wandering” brain state; the device can further include an electronic calendar containing scheduled events, wherein the device includes software that matches each event with an optimal brain state for such event, and means for conveying to the user an optimal brain state when the calendar indicates that the event is imminent or has arrived.

In another aspect, the invention features an electronic device that is programmed to contain audibly or visually accessible multiple brain states which can be accessed by the user either by giving the device a voice command, or by pressing a button on the device, or by pressing an image on a touchscreen of the device; the brain state-conveying prompts can also convey multiple brain states to the user during the course of one day.

In another aspect, the invention features an electronic device that is programmed to contain user-accessible brain states, and conveying to a user one or more of such brain states in response to a device-measured physiological parameter, a user-reported physiological or psychological parameter, a device-determined (e.g., by GPS) or user-reported geographical location, activity of the user, e.g., attendance at a business meeting, or stored calendar or timed events, e.g., a scheduled business meeting.

In another aspect, the invention features an electronic device programmed to match one or more activities with one or more brain states, so that when a user inputs the activity, e.g., a team activity, the device conveys to the user the matching brain state, e.g., “collaborate,” see FIGS. 4-6.

In another aspect, an electronic device is programmed to match one or more office tools with one or more brain states, so that when a user uses the tool, e.g., spreadsheets, the device conveys to the user the matching brain state, e.g., “strategic thinking.”

In another aspect, the invention features an electronic device that includes an electronic calendar containing desirable brain state habit-making experiments, the device being programmed to remind a user to conduct the habit-making experiment; the device can be further programmed to remind the user, at the time of the experiment-conducting reminder, to enter a pre-determined brain state, e.g., meta-awareness.

In another aspect, the invention features an electronic device that includes speech recognition software such that a user can speak to the device regarding an activity or task, e.g., I′m going to spend the next 30 minutes on email processing, and the device will, in response, convey to the user the optimal brain state and strategy for that activity or task, e.g., “Use the thinking brain state and be agile in sequential tasking.”

In various embodiments of the above aspects, the electronic device may include a non-transitory storage medium having instructions that, when executed by a computing platform, result in execution of one or more of the described activities. The device may further include one or more of the following processes: tracking, via a component of the network, data associated with the one or more brain states; and generating, via a component of the network, data associated with the promotion of one or more brain states.

Any of the above aspects may be performed in a data processing system or by a data processing method, e.g., instructional steps carried out by a computer, processor, etc. To illustrate, a diagrammatic system may include, for example, one or more of the following: a processor, a main memory, a static memory, a bus, a video display, an alpha-numeric input device, a cursor control device, a drive unit, a signal generation device, a network interface device, a machine readable medium, instructions and a network. The diagrammatic system may include a personal computer, a handheld device, and/or a data processing system in which one or more operations disclosed herein may be performed. The processor may be a microprocessor, a state machine, an application-specific integrated circuit, a field programmable gate array, etc. The main memory may be a dynamic random access memory and/or a primary memory of a computer system. The static memory may be a hard drive, a flash drive, and/or other memory information associated with the data processing system. The bus may be an interconnection between various circuits and/or structures of the data processing system. The video display may provide graphical representation of information on the data processing system. The alpha-numeric input device may be a keypad (actual or virtual), a keyboard and/or any other input device of text. The cursor control device may be a pointing device such as a mouse, a scroll-wheel, or a virtual pointer. The drive unit may be a hard drive, a storage system, and/or other longer term storage subsystem. The signal generation device may be a bios and/or a functional operating system of the data processing system. The network interface device may be a device that may perform interface functions such as code conversion, protocol conversion and/or buffering required for communication to and from the network. The machine readable medium may provide instructions on which any of the methods disclosed herein may be performed. The instructions may provide source code and/or data code to the processor to enable any one/or more operations disclosed herein.

Although the present aspects have been described with reference to specific examples, it will be evident that various modifications and changes may be made to these aspects without departing from the broader spirit and scope of the various embodiments. For example, the various devices, modules, etc. described herein may be enabled and operated using hardware circuitry, e.g., CMOS based logic circuitry, firmware, software and/or any combination of hardware, firmware, and/or software, e.g., embodied in a machine readable medium.

In several embodiments, the device and/or method steps further includes/utilizes an element for storing data, e.g., a data storage element, where this element is present on an external device, such as a bedside monitor, PDA, smart phone, tablet, computer server, etc. Typically, the data storage element is a computer readable medium. The term “computer readable medium” as used herein refers to any storage or transmission medium that participates in providing instructions and/or data to a computer for execution and/or processing. Examples of storage media include CD-ROM, a hard disk drive, a flash drive (e.g., a USB flash drive), a ROM or integrated circuit, a magneto-optical disk, or a computer readable card such as a PCMCIA card and the like, whether or not such devices are internal or external to the computer.

In another aspect, the invention features a work flow re-engineering system for a team or organization including multiple people, the system including: a) means for using electronic devices for evaluating the existing brain state deployment and brain performance of multiple people as measured by self-report or physiological measure of brain states of the people, correlated with desired outcomes such as productivity, quality, creativity, engagement, or innovation, and b) experimenting with new work flow designs and evaluating brain state deployment and brain performance and desired outcomes using electronic devices until an optimal work flow design has been identified and implemented.

### BRIEF DESCRIPTION OF THE DRAWINGS

FIG. 1 is a representation of a digital watch that prompts the user to engage in a certain brain state.

FIG. 2 is a representation of a mobile smartphone showing a habit strength assessment.

FIG. 3 is a diagram showing a variety of brain states used in daily activities, positioned in two dimensions as a “mind” locator, high to low executive control, and high thinking to high experiencing.

FIGS. 4-5 are drawings depicting the Collaborate brain state and the various brain regions which are highly active (intense orange), moderately active (softer orange), and relatively inactive (blue).

FIGS. 6-9 are drawings depicting the Rapport brain state.

FIGS. 10-11 are drawings depicting the Mind Wandering brain state.

FIGS. 12-14 are drawings depicting the Open Awareness brain state.

FIGS. 15-18 are drawings depicting the Meta Awareness brain state.

FIGS. 19-20 are drawings depicting the Flow brain state.

FIGS. 21-22 are drawings depicting the Narrow Awareness brain state.

### DETAILED DESCRIPTION

The invention, applying principles of neuroscience, psychology, coaching science, and self-science, can employ the following steps:

1. Assess & Set Goals

User assesses current state of brain performance and health, such as productivity, creativity, agility, relational connection, or energy, or psychological measures such as resilience, well-being, or physiological measures such as EEG, heart rate variability or blood pressure, or sociological measures such as social isolation or connectedness. User sets goals to improve brain performance and health, and repeats assessments as progress indicators.

2. Learn

User engages in learning interventions (e.g., listening, watching, reading, assessing, reflecting, discussing) to gain knowledge on the why, what, how, and when considerations for a wide range of potential brain states, brain strategies, and mind habits that have the potential to improve brain performance and health.

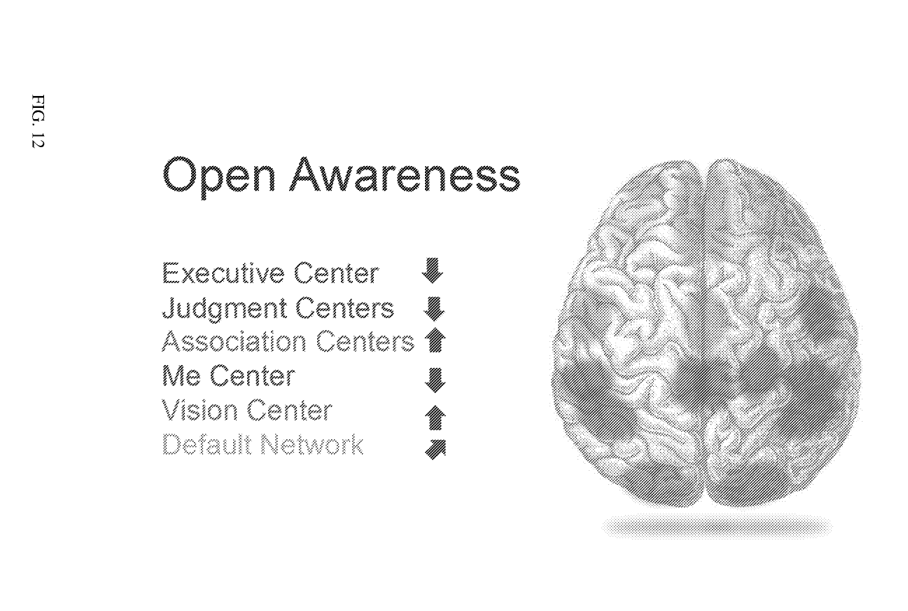

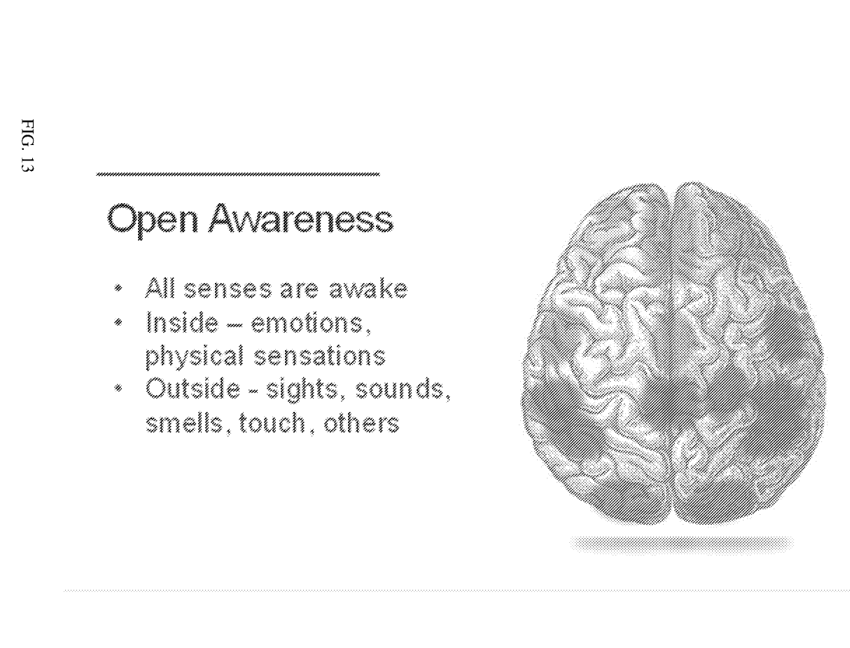

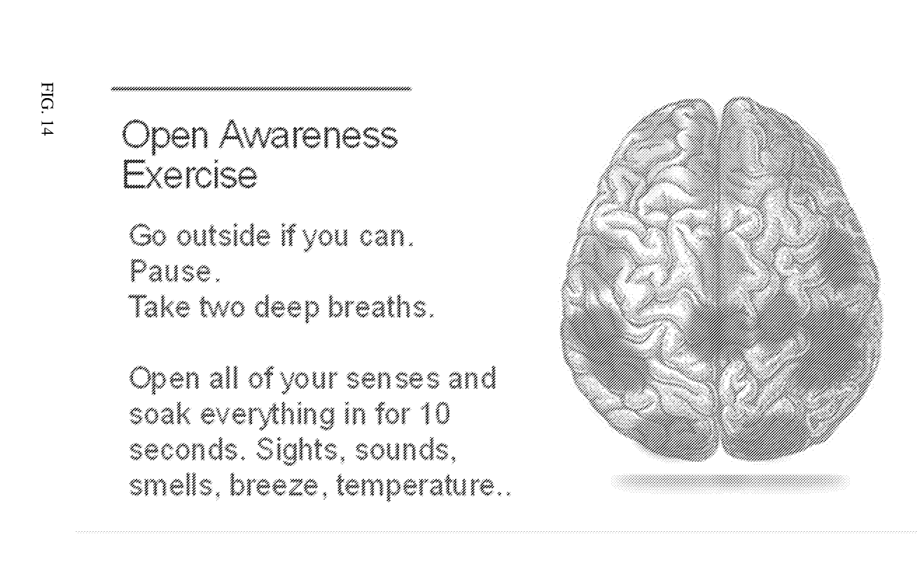

An example of some steps to teach users how to use each brain state is summarized below, using the open awareness brain state as an example (FIGS. 12-14).

a. High and Low Activity Brain Regions

A first step is to learn about brain regions that are active and relatively inactive in a particular brain state, using non-scientific language and metaphor. Let's first consider the low activity areas in the open awareness brain state.

b. Low Activity Brain Regions

In the open awareness state, the mind turns down the activity in the “CEO” or executive center brain region by shifting attentional resources to the senses, such as sight, hearing, touch, and smell. The brain's “judging” region is also turned down so there is no criticism or analysis of the sensory input, just experiencing it as it is. Activity in the “me” or self-referencing center of the brain, which can cause anxiety, is also turned down. Thoughts of oneself, past, present, or future are minimal and ideally absent.

c. High Activity Brain Regions

Now let's explore the active brain regions in the open awareness brain state. Now that the CEO, judging, and me regions of the brain are turned down, the brain is less anxious and has more resources to open up, notice and absorb fully using all of the senses. The brain is able to be present in the moment, experiencing internal and external sensations instead of thinking or judging anything including oneself. The visual, sensory perception, and association centers are activated. The “openness” region of the brain is activated, so one is more open to what's new and different.

d. Recall Past Experiences with Brain State

The next step is to recall times and circumstances of when one uses or has used the open awareness state, perhaps watching a sunset at the beach, or fully experiencing a beautiful day in a park. It is valuable to imagine this brain state and to experience the sensations through imagination. One can observe young children who use the open awareness state frequently, less plagued by a busy mind, and recall younger moments in life. One can also reflect on the conditions which enabled or prevented engaging in this brain state.

e. Experiment and Practice Brain State

Next is experimenting and practice. An exercise would be to walk outside, take a few deep breaths to clear one's mind, then move attention back and down in the brain. Then continue to breathe deeply to relax and take in the full sensory experience of the internal and external worlds. Another exercise is to scan and open each sense, one at a time (seeing, hearing, smelling, touching, tasting) until all senses are awake and alive.

3. Assess, Experiment & Select Habits to Make

For various brain states, brain strategies, and mind habits, user scores a self-assessment or does a physiological assessment, and experiments with potential habits. The experimenting activity leads to the selection of one or more habits worthy of investment in habit-making. Habit-making includes persistent planning, practice, and refinement, and tracking of habit strength and automaticity supported by devices.

4. Habit-Making

User engages a habit-making system of one or more devices to support personalized habit-making—practice, assessment, and refinement, leading to sustainable engagement in new habits as they become stronger and more automatic. Over time a user establishes a habit portfolio of several new brain state and strategy habits during a habit-making phase, which all together improve brain performance and health in a significant and noticeable fashion, and deliver a good return on the investment in habit-making.

The system can include one or more of:
I. Calendar events and alerts, time alerts, geographical locations, or user states, or combinations thereof, are matched, by default or user selection, to optimal brain states, strategies, and instructions, e.g., prompting users to be intentional, undistracted, and go deep in the optimal brain state for a calendar activity, such as the rapport brain state for social conversation, or flow brain state for a solitary creative activity, or narrow awareness brain state for a yoga class. Brain states can also include depictions of the neural networks for certain character strengths or talents; such depictions are valuable to the performance of an activity or response to an event, such as curiosity, compassion, humor, or courage. Prompts may include brain states, any input into the device by the user, graphics, sounds, instructions in written, audio, or video formats, and assessments.
II. An addition to I.: the device prompts the user to address one or more questions, or assess one or more physiological or psychological states, and based upon the answers subsequently prompts user to engage in one or more optimal brain states and follow suggestions or instructions, matched to user's answers or assessments.
III. An addition to I.: a device or multiple devices measure and track over a period of time a user's brain state use, brain activity, physiological state and patterns, e.g. EEG, galvanic skin response, or heart rate or heart rate variability, and prompts user to engage in one or more optimal brain states and follow suggestions or instructions in the moment and/or over time.
IV. An addition to I.: the device can be set to certain modes to support optimal brain performance for a particular activity. For example during a social activity, a “rapport mode” could be activated by default or user selection, encouraging user to turn off or silence device and use certain brain states and strategies without multitasking or distraction.
V. Integration of brain state alerts and instructions or guidelines or suggestions into a suite of office tools deployed on an array of devices, which could include individual or collaborative word processing, email, calendar, databases, publishing, presentations, spreadsheets, and online meeting software. For example, office tools could track user engagement with office tools and:
   a. Add to calendar scheduling software a set of brain state categories, selected by default or user, enabling an event type to be matched to an optimal brain state or series of brain states, and suggestions or instructions.
   b. Match certain brain states and strategies to a tool type (e.g. creative brain states for presentations, strategic brain states for spreadsheets, thinking and agile brain states for email processing, collaborate brain states for online meetings).
   c. Prompt user to deploy brain states well by tracking use and patterns, for example spotting chronic multitasking and suggesting agile sequential tasking, or overuse of one brain state and suggesting diversification to awareness brain states, or reminding user to pause and connect to the intention of the next task in order to upgrade attentional resources.
VI. Scheduled brain state and strategy habit-making prompts to support ongoing experiments, practice, and refinements, by default or selection, as a calendar event or alert. Prompts may include habit descriptions, brain states and strategies, graphics, sounds, instructions in written, audio, or video formats, self-report or physiological assessments.
VII. Engagement of speech recognition software (e.g. voice or avatar on a device including a phone, tablet, game, wearable, game, or robot) to assist in habit-making, e.g. “Hello Avatar, would you help me practice being more agile today? I need to be creative in dealing with a challenging issue,” says Joe. Avatar replies, “Sure Joe, I′ll remind you several times today to practice the agile brain state shifting drill you learned about last week. How about . . . .”
VIII. Engagement of speech recognition software (e.g. voice or avatar on a device including a phone, tablet, wearable, game, or robot), in combination with monitoring of calendar events and/or physiological states, to assist in optimizing brain states, e.g. “Hello Joe, you have an important meeting in 15 minutes and your emotional stress level is a little high. How about a reboot in the open awareness brain state for 3 minutes,” says Avatar.
IX. Calendar items or alerts, time alerts, geographical locations, user physiological or psychological states, speech recognition, or tracking of use of office tools, both current use and patterns, or any combination thereof, random or scheduled, as a prompt to engage a particular brain state. For example:
   a. Pause brain state, for example “meta-awareness:” pause, detach, and observe, objectively in a non-reactive fashion, brain activity such as thoughts, emotions, and behaviors, internal sensations, or the external environment.
   b. Reset or reboot brain state, for example “open awareness:” emptying thinking brain (prefrontal cortex) by moving attention to deeper sensory regions, just as readily performed by a frog or any animal lacking a well-developed prefrontal cortex.
   c. Body intelligence or BQ brain state, for example “body sensory awareness:” emptying thinking brain (prefrontal cortex) by moving attention to body sensory regions and discerning bodily needs.
   d. Mind wandering brain state, which occurs when a user switches off conscious control of attention and allows the mind to wander at will, in order to reduce the risk of mind wandering later during important activities which require a deep focus.
X. Calendar items or alerts, or time alerts, or geographical locations, random or scheduled, to measure physical states by self-report or physiological measures and generate prompts for improvements, such as brain energy and health refuel, via various habits and brain states including exercise, brain foods, hydration, sleep, and brain breaks.
XI. Calendar items or alerts, or time alerts, random or scheduled, to assess emotional states by self-report or physiological measures and generate prompts to practice certain brain states and strategies, such as noticing and naming emotional states, or activating self-compassion or curiosity neural networks, or engaging in social activities, or play/fun activities.
XII. At decision points (e.g. ordering from a menu, selecting from a vending machine, selecting next activity), user seeks help from the device, to prompt engagement in certain brain states and strategies, for example activating the thinking or executive manager brain state and choosing an option aligned with goals, including health, performance, or relationship goals, rather than distractions, impulses or cravings.
XIII. In moments of emotional hijack, impulses, cravings, or distractions, user seeks help from the device to prompt engagement in certain brain states and strategies, to enable a shift from a reactive to a proactive brain state and conscious control of next steps.
XIV. In moments of self-reflection, user seeks help from the device to prompt engagement in certain brain states and strategies to assist in exploring and decoding the messages of emotional states in order to better understand and manage psychological states such as unmet needs, thwarted values, underutilized or underappreciated capacities, or neglected drives.
XV. In social situations, which can disrupt positive habits, user seeks help from the device to prompt engagement in certain brain states and strategies, to assist in staying on track, including the pause or reboot or body sensory brain states mentioned above.
XVI. When adverse events or crises occur, such as external criticism, a health challenge, family illness, or a car breakdown, user seeks help from the device to prompt engagement in certain brain states and strategies which improve coping and resilience.
XVII. When user is struggling to meet personal or professional goals, user seeks help from the device to prompt engagement in certain brain states and strategies that improve persistence and perseverance.
XVIII. For calendar activities or geographical locations or user parameters that require self-coaching, or when user seeks help from the device, the device prompts user to engage in a series of self-coaching brain states and strategies that support learning, change, and growth such as self-reflection, self-awareness, envisioning, insight, motivation, confidence, curiosity, autonomy, self-compassion, and purpose.
XIX. For calendar activities or geographical locations or user parameters that involve coaching others, or when user seeks help from the device, the device prompts user to engage in a series of coaching brain states and strategies which elicit in others various brain states and mindsets that support learning, change, and growth, such as self-reflection, self-awareness, envisioning, insight, motivation, confidence, curiosity, autonomy, self-compassion, and purpose.
XX. For calendar activities or geographical locations or user parameters, or when user seeks help from the device, the device prompts user to engage in a series of self-leadership and leadership brain states and strategies that support optimal performance in self and others, including other-focused, open-minded, and deep listening brain states.

5. Optimal Habit Portfolio

A user or group of users can use the invention to develop a new habit portfolio of brain states and strategies. Brain performance and health may be continually evaluated via self-report measures, such as productivity, creativity, and energy, or physiological measures that correlate with brain health and performance, for example EEG, heart rate variability or blood pressure, as inputs for developing a new habit portfolio well-suited to one's life stage. The development of an optimal brain state and strategy habit portfolio can be done by an individual, family, team, or organization.

6. Optimal Life or Work Structure

A user or group of users can use the invention to re-engineer a new life or work structure, optimizing a mix of activities and brain states each day, week, month or beyond. Brain performance and other performance measures may be continually evaluated via self-report measures, such as productivity, creativity, and energy, or physiological measures that correlate with brain health and performance, for example EEG, heart rate variability or blood pressure, as inputs for testing and changing the mix of activities and brain states. The use of the Organize Your Mind system to re-engineer an optimal life or work structure can be done by an individual, family, team, or organization.

7. Optimal Health & Well-being

A user or group of users can use the invention to improve brain health, and general health and well-being by optimizing a mix of brain states and activities. Brain performance and health may be continually evaluated via self-report measures, such as energy, or physiological measures that correlate with brain health, and general health and well-being, for example EEG, heart rate variability or blood pressure, as inputs for a mix of brain states and activities which improves health and well-being. The use of the Organize Your Mind system to improve brain health, and general health and wellbeing, can be done by an individual, family, team, or organization.

8. Optimal Work Flow

A team, department, or organization can implement the invention to re-engineer work flow. Evaluation of current state of brain state deployment and brain performance, and desired outcome measures such as productivity, quality, engagement, and creativity, can be followed by experimenting with new designs of work flow and brain state use and tests of what combination of activities and brain states and/or sequential series of brain states throughout the work day, week etc, improve brain performance and desired outcomes.

Next is an optimization phase, implementing new work flow designs and brain state use for upgrades to performance and outcomes. Work flow would be customized for different job types such as traders on a stock trading floor, surgical team in an operating room, engineers on a software or device development team, collaborative team of researchers, or a team in charge of manufacturing, customer service, distribution, accounting, or human resources.

9. Optimal Innovation

A team, department, or organization can implement the invention to upgrade innovation—the capacity, quality, speed, and delivery of innovation. Evaluation of the current state of brain state deployment, brain performance, and innovation performance, can be followed by experimenting with new designs of innovation processes. Tests of various combinations of brain states and strategies, and brain state series, deployed over time, can lead to a work flow design which improves innovation in degree, quality, and pace.

For example, new combinations and series of brain state deployment, in a day, week, month or over time, may improve the innovation process by generating more novel ideas, bigger advances or larger leaps from today's constructs, or more effective translation of ideas into testable prototypes, or a more creative refinement and implementation phase. Possibilities include:
a. Frequent and planned engagement in the awareness brain states, or mind wandering states, or nonlinear brain states, or flow states, using optimal strategies, e.g. intentional, deep, agile, diverse, and energized.
b. Frequent and planned engagement in social brain states, including collaborate, rapport, and embodied learning brain states.
c. Implementation of a sequential series of brain states and stategies in a day, work, or over time.

Next are seven examples of individual applications of the invention.

### EXAMPLE 1

### Physician Conducting Patient Visits

Learning & Experimenting Phase

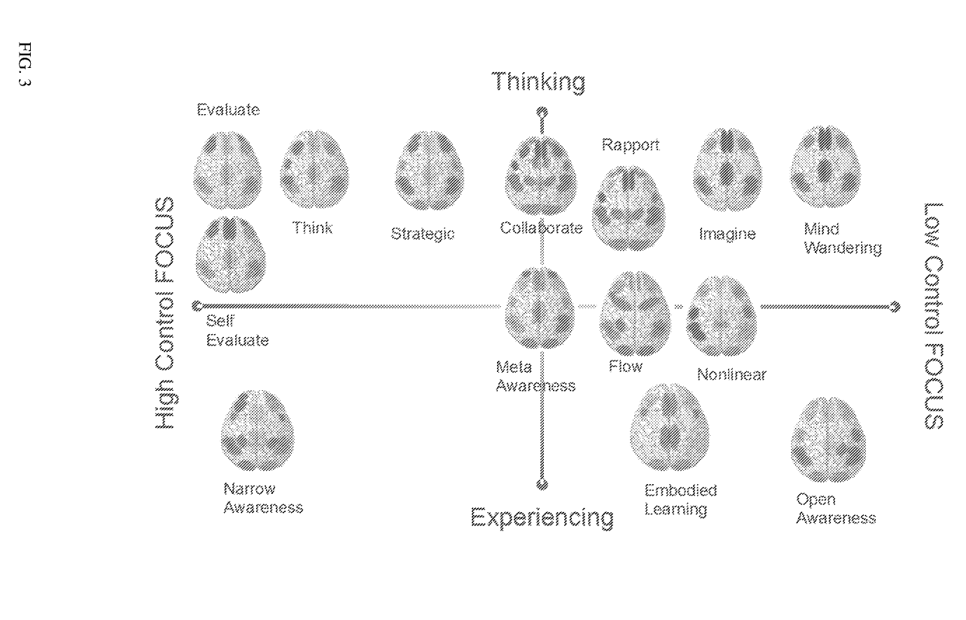

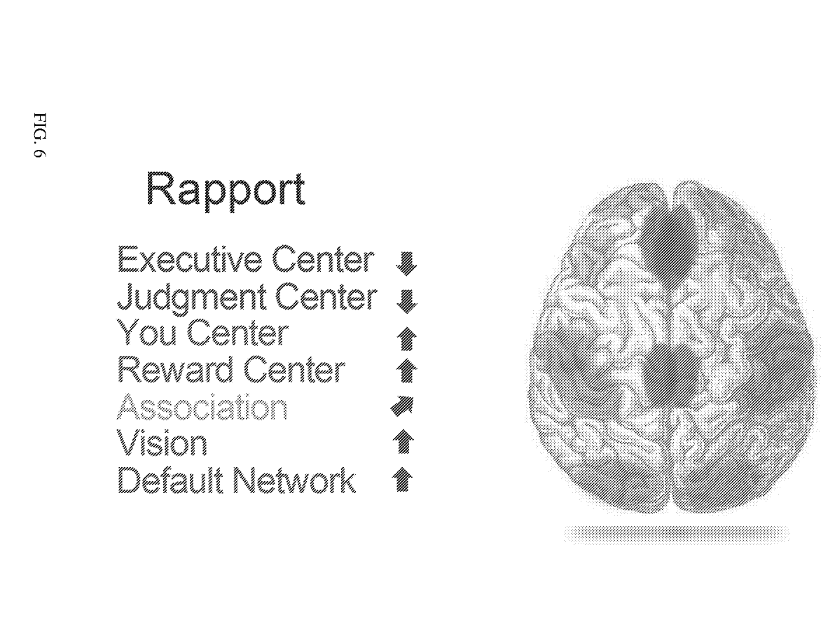

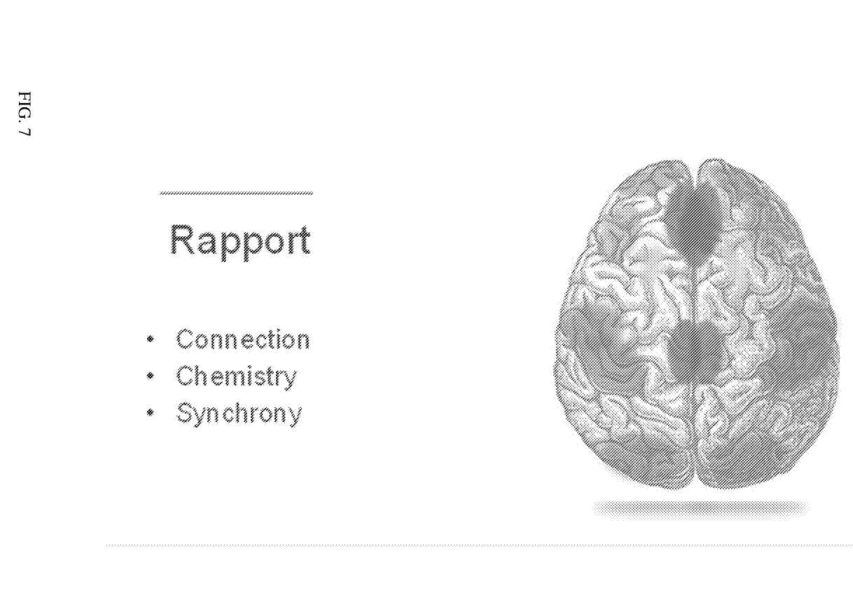

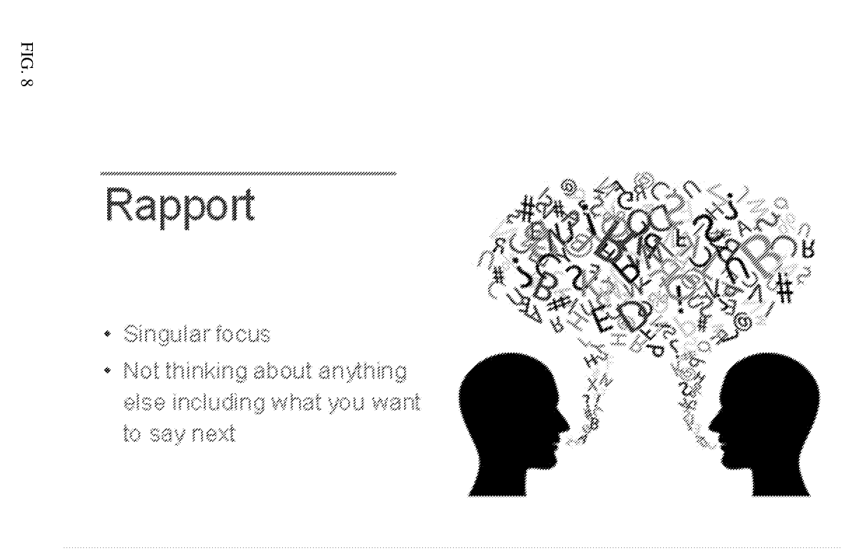

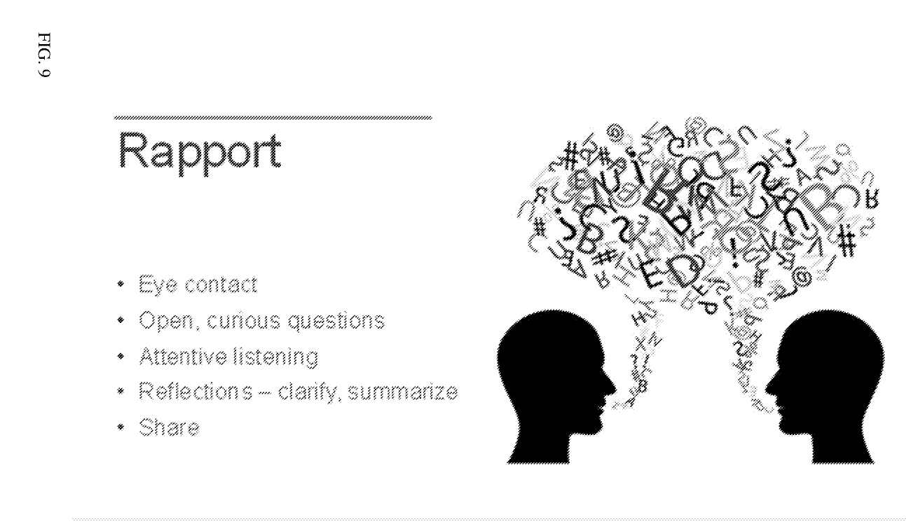

A physician, for a period of three weeks, uses her smartphone to periodically assess (daily or so) how diverse her brain state use was over the past 24 hours, and conducts various experiments help her practice less-used brain states (see FIG. 3). She has discovered that she mainly uses executive control thinking and judging brain states during her busy work days. She is improving her ability to shift into the Rapport brain state (FIG. 6-9) at the beginning of her patient visits. She learns to turn down the highly active brain regions used in her thinking brain states when connecting with a patient. She shifts to the Rapport brain state, so she can be more present and listen more deeply. She discovers that this state is quite creative, as the brain's creative default region is active, and she comes up with interesting new ways to help a patient quickly feel more relaxed and open.

Habit-Making Phase

The physician decides to have her admin team activate the new calendar function on the shared office calendar, matching calendar event types with optimal brain states. Now she gets an alert at the start of each patient visit on her computer, tablet, and phone, to prompt her to shift into the Rapport brain state. The prompt shows the brain state alongside a graphic she chose that has a human face and large heart, to remind her to move her attention out of her fast-thinking, judging brain regions and foster a heartfelt sensory connection. She remembers how creative she can be in helping a patient calm down, a valuable reward that strengthens the habit, which deepens the information exchange and leads to a grateful and engaged patient at the end of the visit.

### EXAMPLE 2

### Overworked Lawyer

Learning & Experimenting Phase

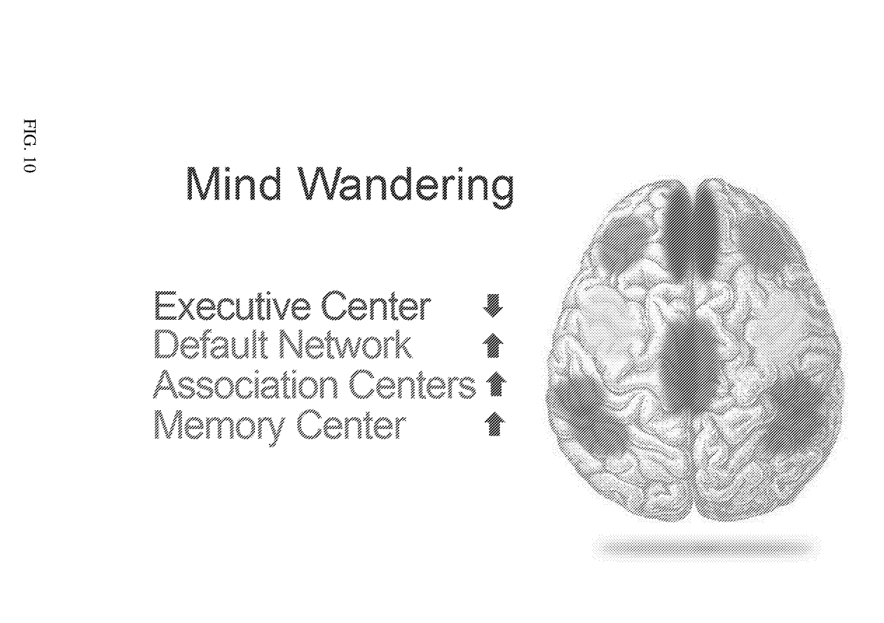

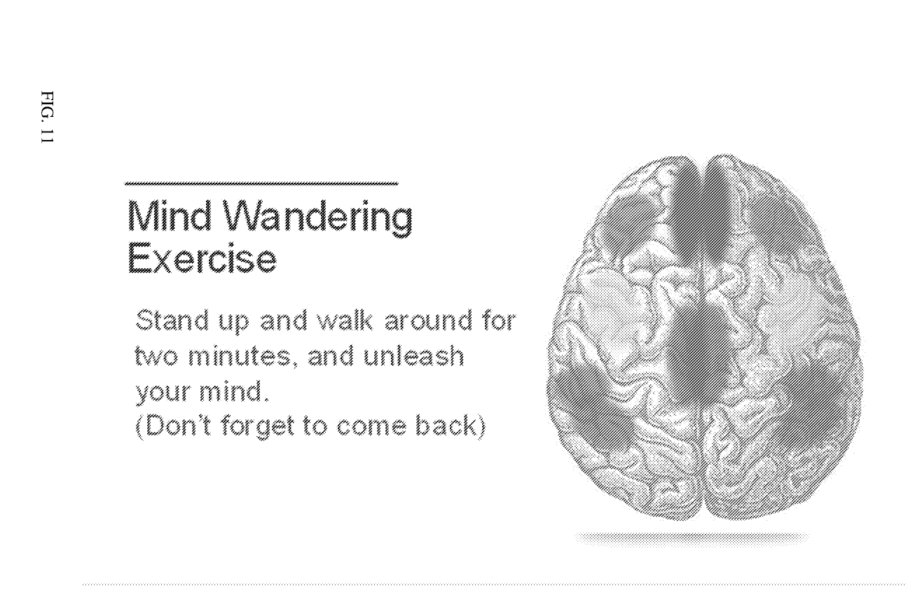

A lawyer, over the course of a month, using his tablet, assesses his brain energy level over the past 24 hours, and conducts various experiments that improve his brain energy. He has discovered that his mind wanders a great deal when working on important legal documents, just when he needs to be deeply focused in his thinking and evaluating brain states. He discovers that when he takes ten minute brain breaks every 90 minutes, and engages the Mind Wandering brain state (FIGS. 10-11) while walking around the block, or climbing stairs in the stairwell, he is able to better sustain a deep focus throughout the day, reducing untimely mind wandering. In the Mind Wandering brain state, when he lets go of controlling his attention, the brain's spontaneous creative (default) centers are active, and his memory works well. His brain's R&D center, the association centers, works beautifully, generating new ideas. He discovers that he has many more new ideas during his mind wandering breaks and he has more physical energy at the end of the day for his family activities. The time investment in brain breaks pays a handsome return.

Habit-Making Phase

The lawyer activates a brain state scheduler on his computer in his digital calendar. The scheduler offers several interesting options: pause or Meta Awareness brain state, reboot or Open Awareness brain state, Body Awareness brain state to tune into what his body needs, or Mind Wandering brain breaks. He has decided to make a brain break habit using the Mind Wandering brain state, at least every two hours.

The brain break alerts display the Mind Wandering brain state and suggest several brief activities that allow his mind to wander nicely, including climbing some stairs, doing some yoga poses his daughter taught him, and walking to a nearby destination and back. Sometimes he ignores the device's prompts for brain breaks, and pays the price later with increased mind wandering and distractibility when he needs deep focus. He has begun to find new kinds of brain breaks, non-important tasks that used to annoy him and can be done while in the mind wandering brain state. When he works from home two days a week, he can make the bed, put in a load of laundry, or empty the dishwasher, tasks that he now looks forward to as brain break activities. His wife is delighted with his new brain break habit.

### EXAMPLE 3

### Frenzied Mom

Learning & Experimenting Phase

A mom, of 10 year old twins, over a couple of months uses her phone to assess how diverse her brain states have been over the past 24 hours. She conducts various experiments that expand her brain's repertoire. She has discovered that she rarely pauses for a sensory experience, savoring her food, noticing a sunset, enjoying cooking, noticing the people around her, or watching the world go by on her porch. She has also noticed that her level of emotional frenzy is often sky-high, making it easy to forget things and feel overwhelmed by her to-do list. She discovers that when she uses the Open Awareness brain state (FIGS. 12-14) as a reboot a few times a day, life feels less overwhelming and she enjoys her kids more. In the Open Awareness brain state, she shifts her attention out of her thinking brain and back and deep into her sensory brain, and sometimes into her chest to experience her breathing. She discovers that she feels quite refreshed after five minutes in the Open Awareness state a few times day. Her kids have been reminding her to do it more often because it puts her in a better mood.

Habit-Making Phase

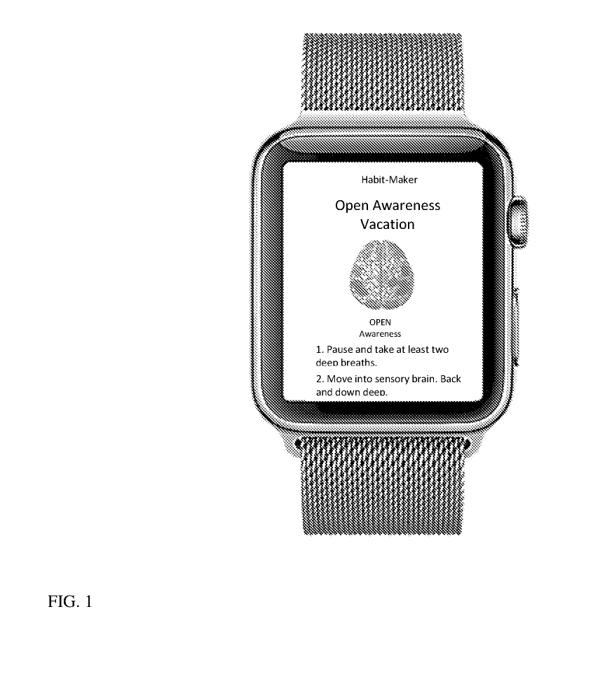

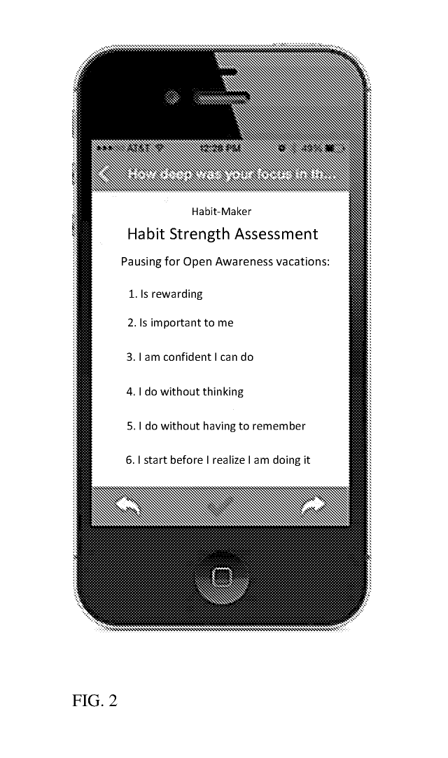

The mom wears a digital watch (see, e.g., FIG. 1) which continually tracks her heart rate variability, a good measure of emotional stress. When her stress rises past a healthy point, she gets an alert to pause, reboot, and shift to an Open Awareness brain state. The alert shows her the brain state and her favorite mountain landscape to activate her senses. At first, the alerts happened 7-8 times a day. Now she catches herself before the alert comes and reboots without the reminder. The surprising outcome is that she is able to be less serious and more playful with her kids and now they are having much more fun together. Her husband appreciates her calmer and lighter mindset. Next she is going to try the family option, where she will get alerts when her kids' emotional stress is high so she can help them calm down quickly. She is wondering whether to have her kids get alerts when her stress is high so that they help her calm down too, making this a family well-being project.

### EXAMPLE 4

### Anxious College Student

Learning & Experimenting Phase

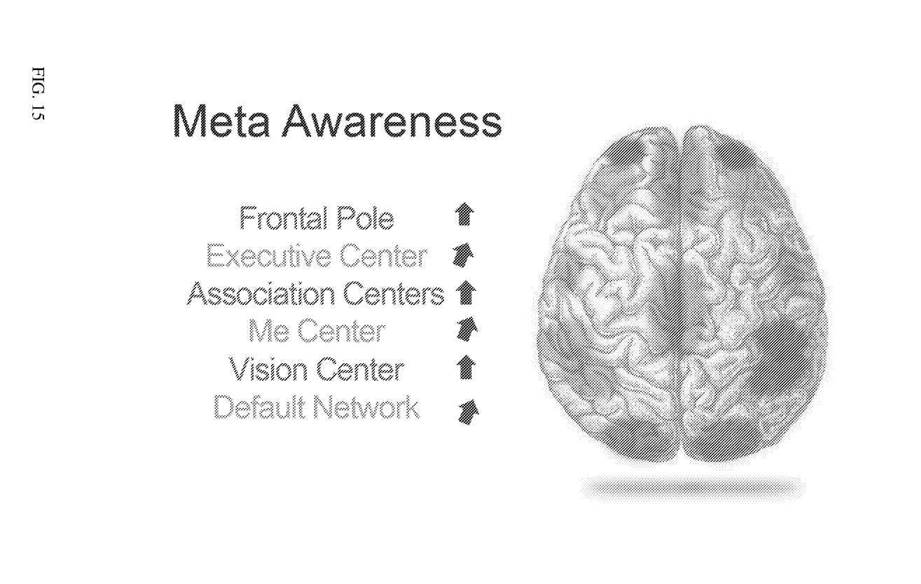

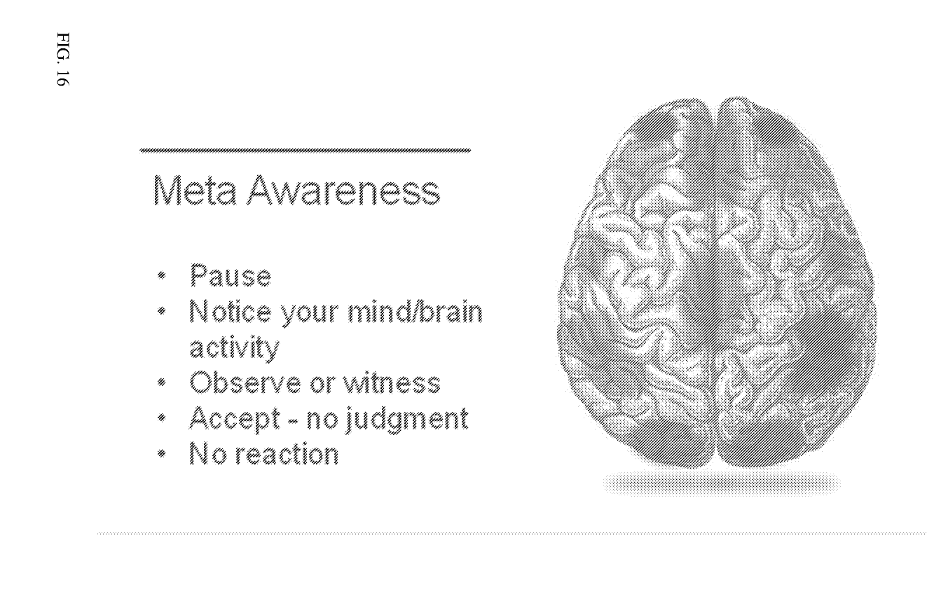

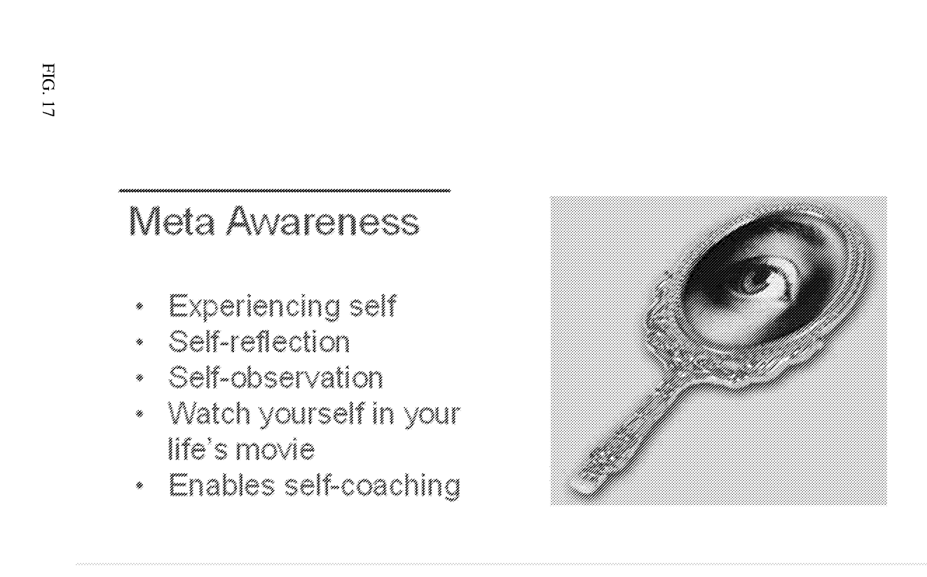

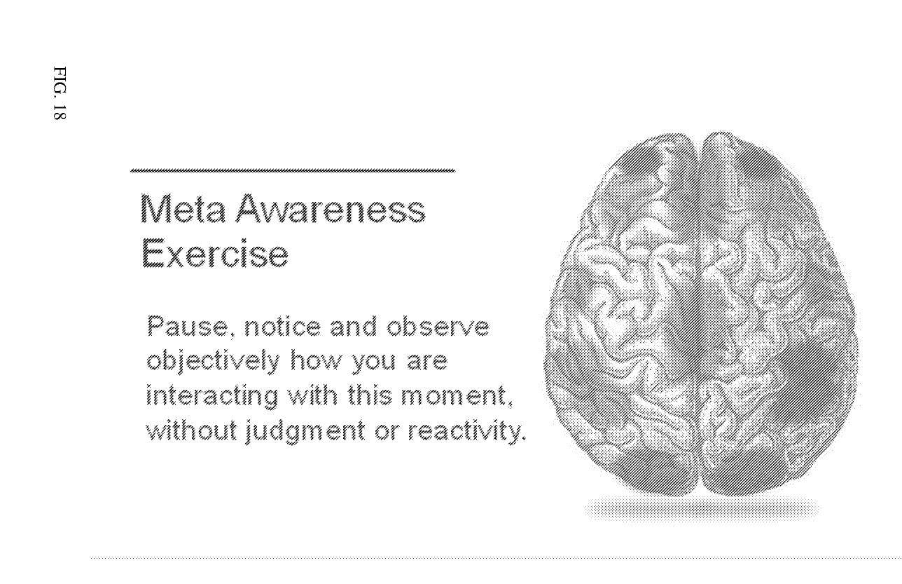

A college student, daily over the course of a week, assesses her emotional energy over the past 24 hours, and conducts various experiments that improve her emotional energy. She discovers that she gets hijacked by her worries in what seems to be a random fashion; it can hit any time of day, and day of the week. Her anxious emotions rush her like a thunderstorm and then it's really hard to pay attention to her schoolwork. She practices the Meta Awareness brain state (FIGS. 15-18), where she activates the region of the brain that enables calm self-observation, as though she is watching her anxious self in her life's movie with curiosity, rather than suffering as the anxious actor in the movie. She learns to notice and name her negative emotions, and she finds that five minutes of stretching on her dorm room carpet calms her down enough to get back to studying. She gets a strategic perspective, realizing that she is worrying more than is warranted some of the time, and can think straight again. When she practices a self-compassion experiment, with her hands crossed over her heart, learning how to suffer well with her anxiety, she calms down even more. What a relief that is.

Habit-Making Phase

The college student has started using the “hijack rescue” button that came installed on her latest phone. When she feels an emotional hijack coming, she clicks on the hijack rescue button and her phone shows her the Meta Awareness state. With a calming voice, her phone walks her through the series of steps that help her settle down. It's working so well that she has shared her experience with some of her friends and now when they get over-excited they all go “Meta” together to calm down together.

### EXAMPLE 5

### Multitasking Manager

Learning & Experimenting Phase

An operations manager at the headquarters of a restaurant chain, daily over the course of a week, assesses how agile he is in shifting brain states between tasks over the past 24 hours. He conducts various experiments that improve his brain's agility. He discovers that he multitasks at such a fast rate that he isn't agile—he keeps thinking, even fretting, about the last task and future tasks when he is trying to focus on the present task. He sometimes forgets things, makes mistakes, and is completely exhausted at the end of the day. He also doesn't feel as though he is getting traction on his most challenging projects.

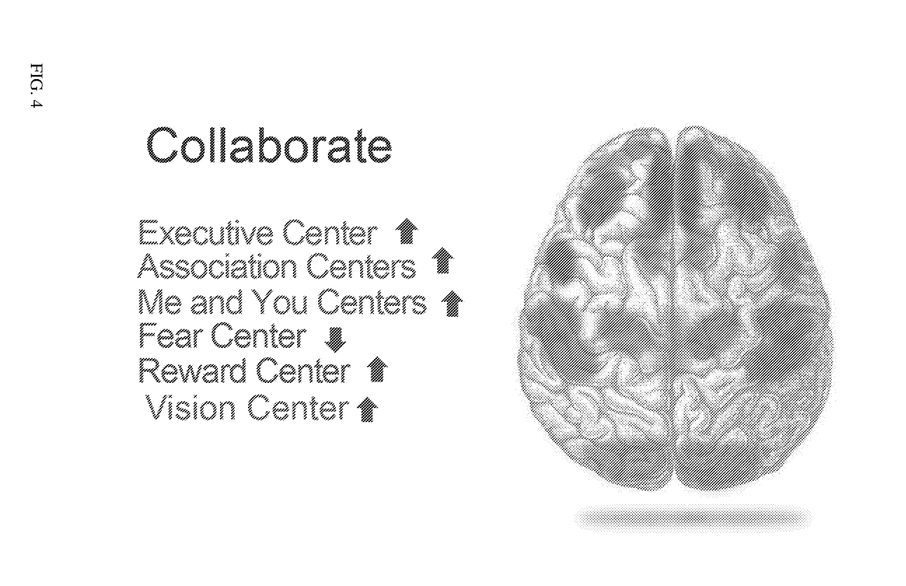

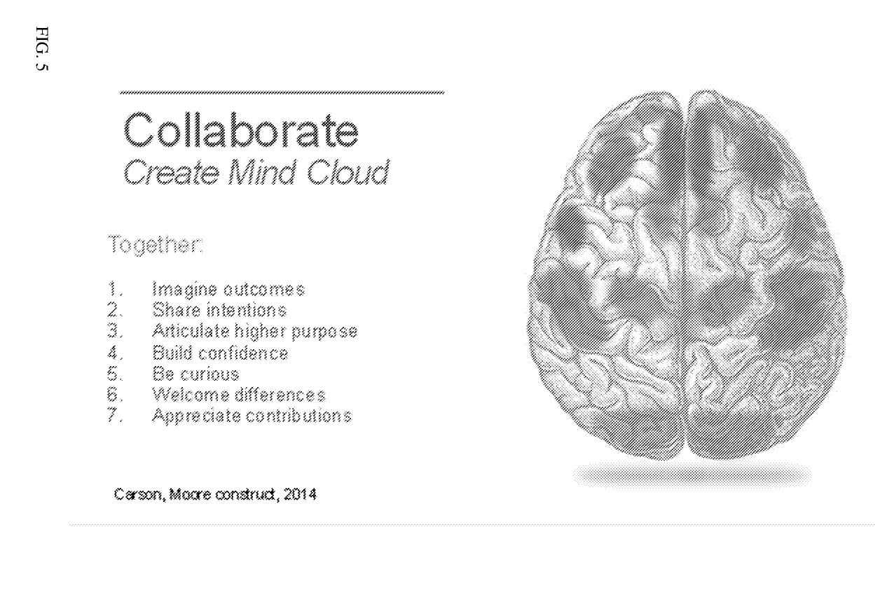

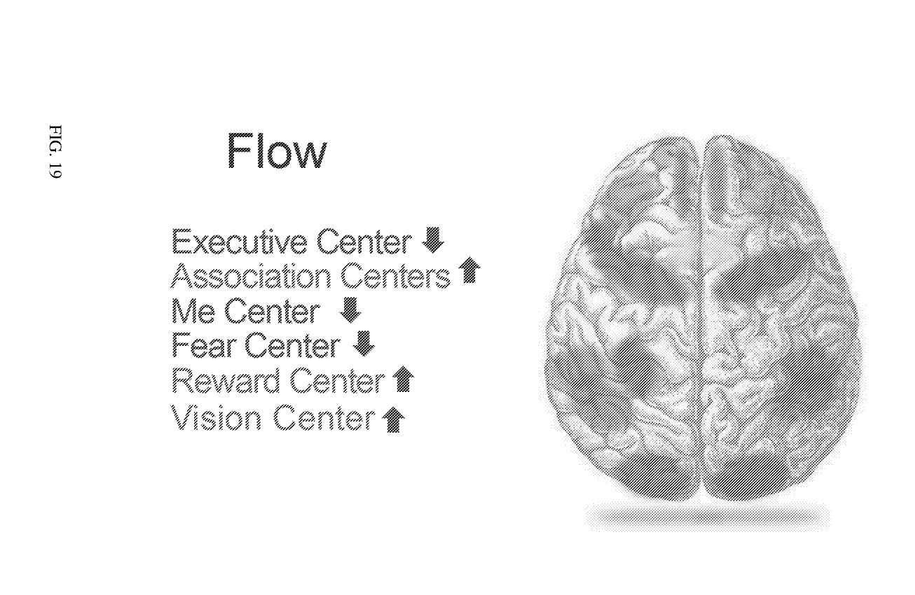

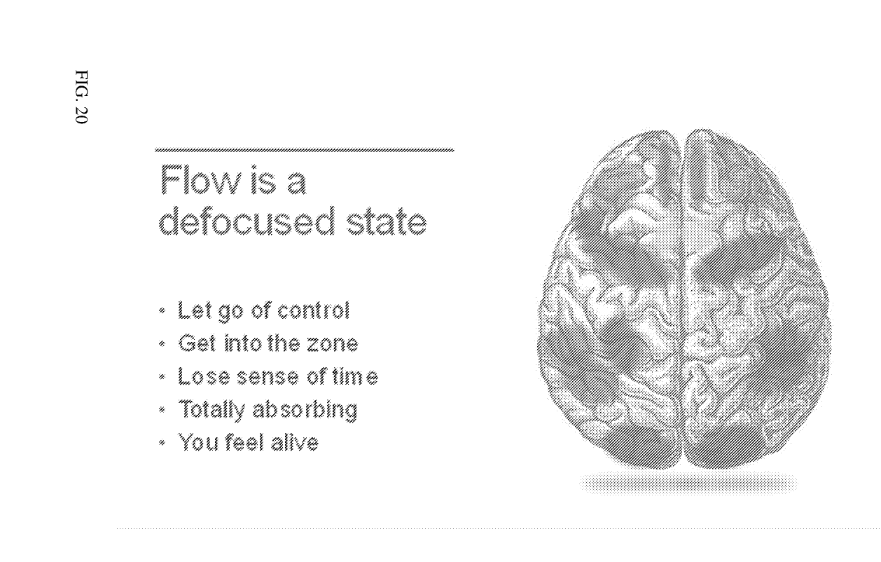

He practices an agile brain state shifting drill where he shifts from being deeply in one brain state, say the Rapport brain state (FIGS. 6-9) and a conversation with a colleague, into deep focus on a creative project or the Flow brain state (FIGS. 19-20). Rather than scattering attention over several tasks he is starting to appreciate the value of deep focus and agile, sequential tasking. The big upside comes in team meetings as he has taught his team to go deep into one focus and then shift with agility into the next topic. They all turn off their devices to avoid distractions and multitasking, and their meetings are done in half the time they used to be when everyone was multitasking. Their team relationships are deepening too as they fully use the Collaborate brain state (FIGS. 4-5). In fact they almost always come up with some useful new ideas, have more laughs, and are energized at the end of meetings instead of fried.

Habit-Making Phase

The manager has selected two approaches to help him with deep focus on tasks followed by agile task shifting and sequential tasking. He is finding that multitasking is a hard habit to change. He wonders whether he is addicted to his devices. First he activates the function in his office tools that tracks how many windows he has open on his computer at one time and prompts him to close everything but the windows he needs for deep focus on his current task. The prompt also shows the Flow brain state, a deep and creative state which he just learned is vital for psychological well-being. He also activates the “flow” mode on his phone so that it displays the Flow brain state and shuts off the phone so he can't multitask and access any of its functions while he is engaged in a creative project. It's such rewarding work that over a few weeks he loses some of his craving for multitasking. He had also been getting worried that he was becoming addicted to his smartphone and now he has hope that he can cope without it, even for a few hours at a time.

### EXAMPLE 6

### Gaming-Obsessed Teenager

Learning & Experimenting Phase

A 14 year old teenage boy spends five hours or more at a time playing the latest, coolest online games, slouching in front of his screen, forgetting about his homework and family activities, and eating junk food because it is fast and can be consumed without missing a beat. His parents are concerned about how his game obsession might be rewiring his brain in ways that may not be helpful to his transition through a college education and into responsible adulthood. They fret even more when they think about the unhealthy aspects of long sedentary periods, sleep disruption, and unhealthy nutrition. Yikes . . . .

The teenager has moments of doubt about how his current lifestyle is affecting his social life and ability to attract girls, and an occasional flicker of concern about his high school grades. He reads an ad on the newest game about a “brain upgrade” feature. The game comes with a wearable EEG sensor (an EEG-measuring band that wraps around his head) and it tracks his brain activity while he plays the game. The sensor gives him advice on which brain state to use for which game stage, and even tells him when his game would be improved if he were to go use his body for 15 minutes, shooting some hoops, or running around the block. Even diversifying to a 30-minute high control thinking episode focused on his homework could potentially up his game performance.

Habit-Making Phase

The teenager buys the brain upgrade feature, of course not telling his parents as he is in charge of his own life and set up a secret Paypal account. It's very cool to learn about how the brain works in these various brain states and what strategies improve his game performance. He is finding that when he eats a healthy dinner and drinks water and not coke, he has more brain energy. What really surprised him is that brain breaks to do homework and exercise have taken him to a whole new level of game performance. Don't tell his parents but the girls are impressed with his smarts around how to use the brain well. He is practicing a deep focus in the Rapport brain state (FIGS. 6-9) which makes them smile warmly and openly. The Collaborate brain state (FIGS. 4-5) makes him a better basketball player and he gets more cheers from girls. He's beginning to think of himself as a dude instead of a nerd.

### EXAMPLE 7

### Widower Dealing with an Aging Brain

Learning, Experimenting & Habit-Making

A 75 year old widower is getting tired of hints from his kids that he should start considering a move into an assisted living residence. He already down-sized after his wife died of breast cancer, and he treasures his independence. He bumped into an old friend at the local donut shop and they commiserated about how adult kids don't have a clue what it's like to walk in their shoes. His friend told him about a robot he bought six months ago named Indy, short for Independence. Indy helped his friend get healthier and happier, and get a spring in his step that he hadn't experienced for a while.

The widower went online and found out that Indy came with a money-back guarantee so he broke his frugal habit and bought the robot. First Indy talked to him about his brain fog, his eating habits, and his diabetes. Indy showed him what the brain looks like when it is well-nourished and poorly nourished. He cut down on donuts and followed Indy' s suggestions to eat complex carbs, lean protein, veggies or fruits, at most meals. Amazingly with Indy' s reminders and cooking advice his brain fog is gone, except when he eats donuts which are no longer a daily habit but a weekly treat.

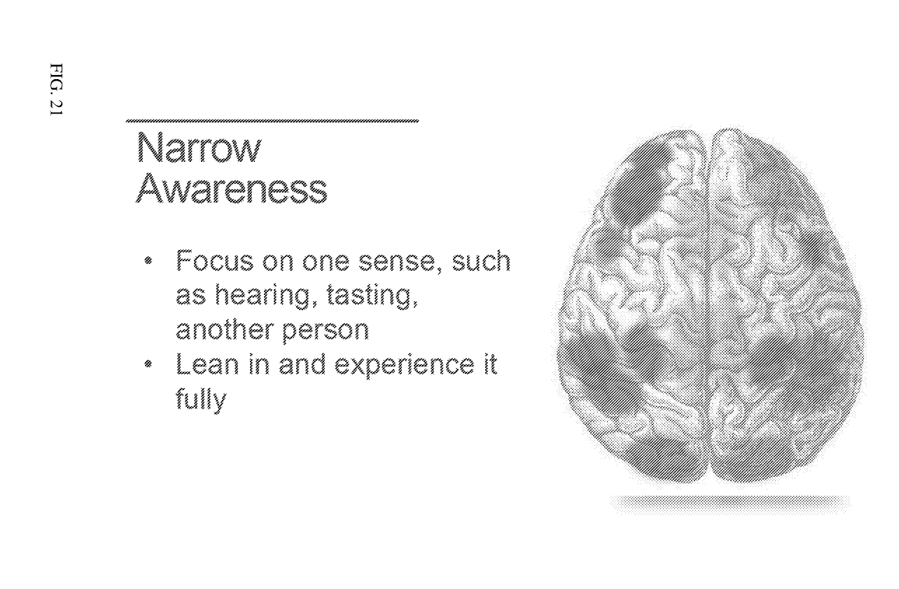

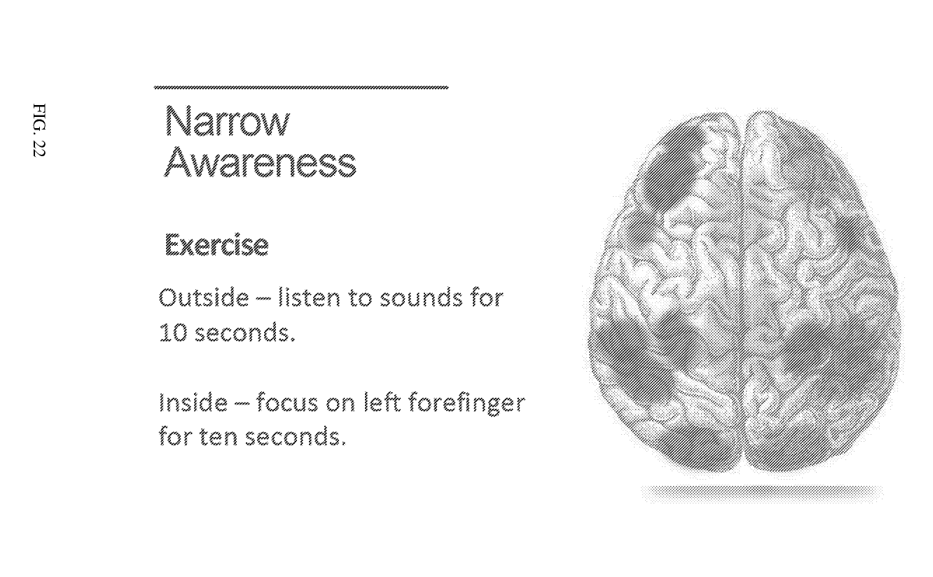

Next Indy showed him pictures of the brain with and without exercise and explained that exercise would slow aging and help his brain grow. Indy taught him to use the Narrow Awareness brain state (FIG. 21-22) when exercising so that he would be careful not to injure his muscles. The most interesting thing Indy described is the brain regions that are activated when people connect with a higher purpose, and that more purposeful experiences might improve his brain performance. He began to call someone most days—a friend in need, or one of his grandkids who was struggling. Indy reminded him to make the calls every morning. He learned how to use the Rapport brain state (FIGS. 6-9) and offer support. Now that made him feel good. He began to look for other people to go for walks with him, people who needed cheering up.

Indy is a curious robot, always asking him questions about what is new in his life. Indy taught him that loss of curiosity is one of the warning signs of Alzheimer's disease, and showed him the curious brain state. Now he looks for new things in the daily paper and tries to be more curious in his phone calls. Amazingly he has gotten better at remembering names.

Indy has started to talk to him about learning a little Spanish so he can converse in Spanish a little with his Hispanic daughter-in-law, and their upcoming family trip to Mexico. He has the energy to socialize more and wants to find a female companion. Best of all, his kids have stopped talking about assisted living.

### Other Embodiments

All publications, patents, and patent applications mentioned in the above specification are hereby incorporated by reference. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the claims.

1. An electronic device that comprises an electronic calendar containing scheduled events such as meetings, exercise sessions, social events, and medical appointments, said device comprising:
a) software that matches an event with an optimal brain state for such event, and
b) means for conveying to the user of the device said optimal brain state when the calendar indicates an event reminder or that the event is imminent or has arrived.

2. The device of claim 1, wherein said device comprises a screen, and said optimal brain state is displayed visually on said screen.

3. The device of claim 1, wherein said device comprises an audible function, and said device conveys to the user an audible prompting signal when the calendar indicates that the event is imminent or has arrived.

4. The device of claim 3, wherein said signal includes a verbal identification of said optimal brain state.

5. The device of claim 1, wherein said device includes means, e.g., a keyboard or touchscreen, whereby the user can input one or more of the following: i) an assessment, e.g., on a one-to-five scale, of the user's real-time physical energy or health state, ii) an assessment, e.g., on a one-to-five scale, of the user's real-time emotional state, and iii) an assessment, e.g., on a one-to-five scale, of the user's real-time cognitive performance, e.g., EEG, focus, mental agility, creativity, and working memory.

6. The device of claim 1, wherein said device includes means for physically measuring a biological parameter, e.g., EEG, heart rate variability or blood pressure, that varies with one or more physical or emotional states, or brain performance or health, of the user, and wherein said device comprises means for conveying to said user recommended actions to improve said physical or emotional states, or brain performance or health.

7. The device of claim 6, wherein said conveying means includes audible cues.

8. The device of claim 6, wherein said conveying means includes a screen on which can be displayed said recommended actions.

9. The device of claim 1, wherein said device is a phone.

10. The device of claim 1, wherein said device is a watch.

11. The device of claim 1, wherein the device is an electronic tablet.

12. The device of claim 1, wherein the device is a robot.

13. The device of claim 1, wherein the device is a wearable device.

14. The device of claim 1, wherein the device is a game device.

15. The device of claim 1, wherein the device is further programmed to aid said user in practicing a selected desirable habit by i) conveying to said user prompts, at pre-determined intervals, or based on geographic or user metric triggers, to practice said habit, ii) tracking the practicing of said selected habit, iii) tracking the rewards flowing from the practicing of said selected habit, and iv) using the tracking of step iii) to calculate the degree to which the habit becomes automatic.

16. The device of claim 15, wherein the device is programmed to receive and store a goal of said user, attainment of which is aided by practicing and keeping said habit.

17. The device of claim 15, wherein said device is programmed to present to the user a series of experiments regarding one or more desirable habits, and to store the results of said experiments.

18. The device of claim 15, wherein said brain state-conveying prompts convey multiple brain states to the user during the course of one day.

19. An electronic device that is programmed to provide visual or audible prompts to the user of the device, said prompts occurring either: a) randomly throughout the day, or b) at predetermined times throughout the day, or c) on user request, wherein said prompts convey to the user a predetermined brain state such as meta awareness (a “pause” brain state), open awareness (a “reboot” brain state), BQ (a “body sensory awareness” brain state) or a “mind wandering” brain state.

20. The device of claim 19, wherein the device further comprises an electronic calendar containing scheduled events, and wherein the device comprises software that matches an event with an optimal brain state for such event, and means for conveying to the user said optimal brain state when the calendar indicates that the event is imminent or has arrived.

21. An electronic device that is programmed to contain audibly or visually accessible multiple brain states which can be accessed by the user either by giving the device a voice command, or by pressing a button on the device, or by pressing an image on a touchscreen of the device.

22. An electronic device that is programmed to contain user-accessible brain states, said device being capable of conveying to a user one or more of such brain states in response to a device-measured physiological parameter, a user-reported physiological or psychological parameter, a device-determined (e.g., by GPS) or user-reported geographical location, activity of the user, e.g., attendance at a work meeting, or stored calendar or timed events, e.g., a scheduled meeting with a colleague.

23. An electronic device programmed to match one or more activities with one or more brain states, so that when a user inputs the activity, e.g., a team activity, the device conveys to the user an optimal brain state, e.g., “collaborate.”

24. An electronic device programmed to match one or more office tools with one or more brain states, so that when a user inputs the tool, e.g., spreadsheets, the device conveys to the user an optimal brain state, e.g., “strategic thinking.”

25. An electronic device that comprises an electronic calendar containing desirable habit-making experiments, said device being programmed to remind a user to conduct the habit-making experiment.

26. The device of claim 25, wherein the device is further programmed to remind the user, at the time of the experiment-conducting reminder, to enter a pre-determined brain state, e.g., meta-awareness.

27. An electronic device comprising speech recognition software such that a user can speak to the device regarding an activity or task, e.g., writing an article, and the device will, in response, convey to the user the optimal brain state or strategy for that activity or task, e.g., “nonlinear brain state and agile strategy.”

28. A work flow re-engineering system for a team or organization including multiple people, said system comprising: a) means using electronic devices for evaluating the existing brain state deployment and brain performance of multiple people as measured by self-report or physiological measure of brain states of said people, correlated with desired outcomes such as productivity, quality, creativity, engagement, or innovation, and b) experimenting with new work flow designs and evaluating brain state deployment and brain performance and desired outcomes until an optimal work flow design has been identified and implemented.
